(19) United States
(12) Patent Application Publication
(10) Pub. No.: US 2024/0374820 A1
(43) Pub. Date: Nov. 14, 2024

(54) INFORMATION PROCESSING METHOD, INFUSION PUMP, INFORMATION PROCESSING APPARATUS, COMPUTER PROGRAM, AND INFORMATION PROCESSING SYSTEM

(71) Applicant: TERUMO KABUSHIKI KAISHA, Tokyo (JP)

(72) Inventor: Shohei SASAKI, Chigasaki-shi (JP)

(73) Assignee: TERUMO KABUSHIKI KAISHA, Tokyo (JP)

(21) Appl. No.: 18/777,196

(22) Filed: Jul. 18, 2024

Related U.S. Application Data
(63) Continuation of application No. PCT/JP2023/003301, filed on Feb. 2, 2023.

(30) Foreign Application Priority Data
Feb. 3, 2022 (JP) 2022-015804

(51) Int. Cl. A61M 5/172; A61M 5/142; G16H 40/40

(52) U.S. Cl. CPC A61M 5/172; G16H 40/40; A61M 2005/14208; A61M 2205/18; A61M 2205/50

(57) ABSTRACT

An information processing method, an infusion pump, an information processing apparatus, a computer program, and an information processing system are provided for predicting a failure in an infusion pump. A duty ratio of a voltage to be applied to a motor included in the infusion pump that delivers liquid delivery while maintaining a constant flow rate is recorded, whether or not there is a risk of occurrence of a failure in the infusion pump is determined based on a history of the duty ratio, and, in a case in which there is a risk of occurrence of a failure, prediction of occurrence of the failure is output.

### CROSS-REFERENCE TO RELATED APPLICATIONS

This is a bypass continuation of PCT Application No. PCT/JP2023/003301, filed on Feb. 2, 2023, which claims priority to Japanese Application No. 2022-015804, filed on Feb. 3, 2022. The entire contents of these application are incorporated by reference herein.

### BACKGROUND

The present disclosure relates to an information processing method, an infusion pump, an information processing apparatus, a computer program, and an information processing system for performing processing related to operation of an infusion pump.

As a drug therapy for cancer pain, a patient controlled analgesia (PCA) method is known. The PCA method is a method in which a patient appropriately administers a medicinal solution such as an analgesic by his/her own operation. In the PCA method, a portable infusion pump that delivers liquid such as a medicinal solution is used. The infusion pump is connected to an injection needle inserted into the body of the patient, the infusion pump delivers a medicinal solution, and the medicinal solution is injected into the body of the patient through the injection needle. Japanese Patent Publication No. 2015-516207 A describes an example of the infusion pump.

### SUMMARY

There is a possibility that a failure will occur in an infusion pump during operation. For example, a failure may occur as a result of a movable portion wearing, and wear powder being clogged in the movable portion. If a failure occurs while the patient is using the infusion pump, the patient is adversely affected. In addition, repairing the infusion pump in which a failure has occurred imposes a heavy burden on a user such as a patient or a medical worker. To avoid occurrence of a failure, it is necessary to perform maintenance such as periodic inspection and replacement of deteriorated parts. However, performing appropriate maintenance imposes a heavy burden on the user. If a failure of the infusion pump can be predicted, maintenance such as replacement of a part before the failure occurs can be efficiently performed.

Embodiments of the present disclosure have been developed in view of such circumstances, and an object of certain embodiments is to provide an information processing method, an infusion pump, an information processing apparatus, a computer program, and an information processing system for easily predicting a failure of an infusion pump.

An information processing method according to an embodiment of the present invention includes recording a duty ratio of a voltage to be applied to a motor included in an infusion pump that delivers liquid while maintaining a constant flow rate, determining whether or not there is a risk of occurrence of a failure in the infusion pump based on a history of the duty ratio, and outputting prediction of occurrence of a failure in a case in which there is a risk of occurrence of the failure.

An infusion pump according to an embodiment of the present invention is an infusion pump that delivers liquid while maintaining a constant flow rate, the infusion pump including a motor that operates to deliver liquid, a recording unit that records a duty ratio of a voltage to be applied to the motor to operate the motor, a determination unit that determines whether or not there is a risk of occurrence of a failure in the infusion pump based on a history of the duty ratio, and an output unit that outputs prediction of occurrence of a failure in a case in which there is a risk of occurrence of the failure.

An information processing apparatus according to an embodiment of the present invention includes an acquisition unit that acquires a duty ratio of a voltage to be applied to a motor included in an infusion pump that delivers liquid while maintaining a constant flow rate, from the infusion pump, a recording unit that records the acquired duty ratio, a determination unit that determines whether or not there is a risk of occurrence of a failure in the infusion pump based on a history of the duty ratio, and an output unit that outputs prediction of occurrence of a failure in a case in which there is a risk of occurrence of the failure.

A computer program according to an embodiment of the present invention causes a computer to execute processing of acquiring a duty ratio of a voltage to be applied to a motor included in an infusion pump that delivers liquid while maintaining a constant flow rate, from the infusion pump, recording the acquired duty ratio, determining whether or not there is a risk of occurrence of a failure in the infusion pump based on a history of the duty ratio, and outputting prediction of occurrence of a failure in a case in which there is a risk of occurrence of the failure.

An information processing system according to an embodiment of the present invention includes an infusion pump and an information processing apparatus, in which the infusion pump includes a motor that operates to deliver liquid and transmits a duty ratio of a voltage to be applied to the motor to operate the motor, to the information processing apparatus, and the information processing apparatus receives the duty ratio, records the received duty ratio, determines whether or not there is a risk of occurrence of a failure in the infusion pump based on a history of the duty ratio, and outputs prediction of occurrence of a failure in a case in which there is a risk of occurrence of the failure.

In one embodiment of the present invention, the infusion pump delivers liquid while maintaining a constant flow rate, and it is determined whether or not there is a risk of occurrence of a failure in the infusion pump based on the duty ratio of the voltage to be applied to the motor of the infusion pump. In the infusion pump, load of the motor increases or decreases in accordance with deterioration, and the duty ratio changes, so that it is possible to determine whether or not there is a risk of occurrence of a failure based on the history of the duty ratio. It is therefore possible to easily predict occurrence of a failure in the infusion pump before the failure occurs.

The present disclosure provides beneficial effects such as making maintenance of the infusion pump more efficient than in the related art, and making it possible to avoid occurrence of a failure of the infusion pump in advance.

### BRIEF DESCRIPTION OF THE DRAWINGS

FIG. 1 is a schematic view illustrating a usage example of an infusion pump according to a first embodiment.

FIG. 2 is a block diagram illustrating an example of an internal functional configuration of the infusion pump according to the first embodiment.

FIG. 3 is a schematic view illustrating a configuration example of a liquid delivery mechanism.

FIG. 4 is a graph indicating temporal change of a voltage to be applied to a motor.

FIG. 5 is a graph schematically indicating an example of temporal change of a duty ratio.

FIG. 6 is a flowchart indicating an example of procedure of processing to be executed by the infusion pump according to the first embodiment.

FIG. 7 is a schematic view illustrating an output example of prediction of occurrence of a failure.

FIG. 8 is a schematic view illustrating a configuration example of an information processing system according to a second embodiment.

FIG. 9 is a block diagram illustrating an example of an internal functional configuration of an infusion pump according to the second embodiment.

FIG. 10 is a block diagram illustrating an internal configuration example of an information processing apparatus according to the second embodiment.

FIG. 11 is a flowchart indicating an example of procedure of processing to be executed by the information processing system according to the second embodiment.

FIG. 12 is a flowchart indicating an example of procedure of processing to be executed by the information processing system according to the second embodiment.

FIG. 13 is a block diagram illustrating an internal configuration example of an information processing apparatus according to a third embodiment.

FIG. 14 is a conceptual diagram illustrating functions of a learned model.

FIG. 15 is a flowchart indicating an example of procedure of processing to be executed by an information processing system according to the third embodiment.

### DETAILED DESCRIPTION

Hereinafter, the present invention will be specifically described with reference to the drawings illustrating embodiments of the present invention.

### First Embodiment

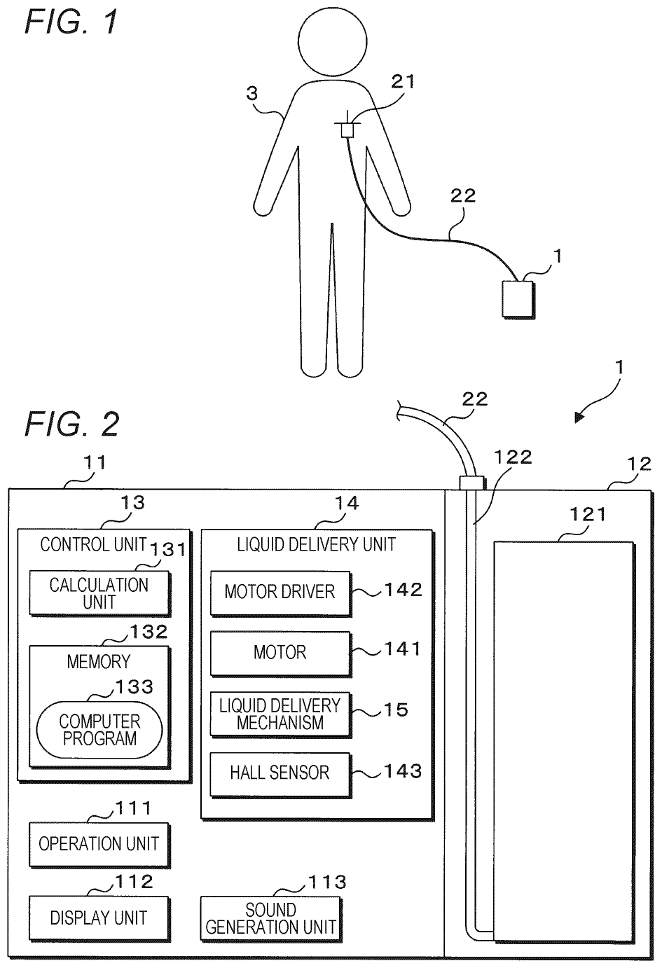

FIG. 1 is a schematic view illustrating a usage example of an infusion pump 1 according to a first embodiment. The infusion pump 1 is used as a PCA pump to be used in a PCA method. An injection needle 21 is inserted into the body of a patient 3, and the infusion pump 1 is connected to the injection needle 21 via an infusion tube 22. When a user such as the patient 3 or a medical worker operates the infusion pump 1, the infusion pump 1 delivers a medicinal solution, and the medicinal solution is administered into the body of the patient 3 through the infusion tube 22 and the injection needle 21. For example, a size of the infusion pump 1 is a size that can be carried by the patient 3 with one hand.

FIG. 2 is a block diagram illustrating an example of an internal functional configuration of the infusion pump 1 according to the first embodiment. The infusion pump 1 includes a pump body 11 and a cartridge portion 12. The cartridge portion 12 is attachable to and detachable from the pump body 11. The cartridge portion 12 includes a tank 121 and a tube 122. The tank 121 stores a medicinal solution. The tube 122 has one end connected to the tank 121 and the other end connected to the infusion tube 22. The medicinal solution stored in the tank 121 is delivered to the infusion tube 22 through the tube 122.

The pump body 11 includes a control unit 13, a liquid delivery unit 14, an operation unit 111, a display unit 112, and a sound generation unit 113. The control unit 13 includes a calculation unit 131 and a memory 132. The calculation unit 131 comprises, for example, a central processing unit (CPU), a graphics processing unit (GPU), or a multi-core CPU. The calculation unit 131 may comprise a quantum computer. The memory 132 is a non-volatile memory. The control unit 13 may further include a volatile memory. The memory 132 stores a computer program 133 and stores various kinds of information necessary for the calculation unit 131 to execute information processing. The control unit 13 executes various kinds of processing by the calculation unit 131 executing information processing according to the computer program 133. The control unit 13 controls operation of each unit of the pump body 11. The control unit 13 may comprise hardware that does not store the computer program 133, such as a field programmable gate array (FPGA).

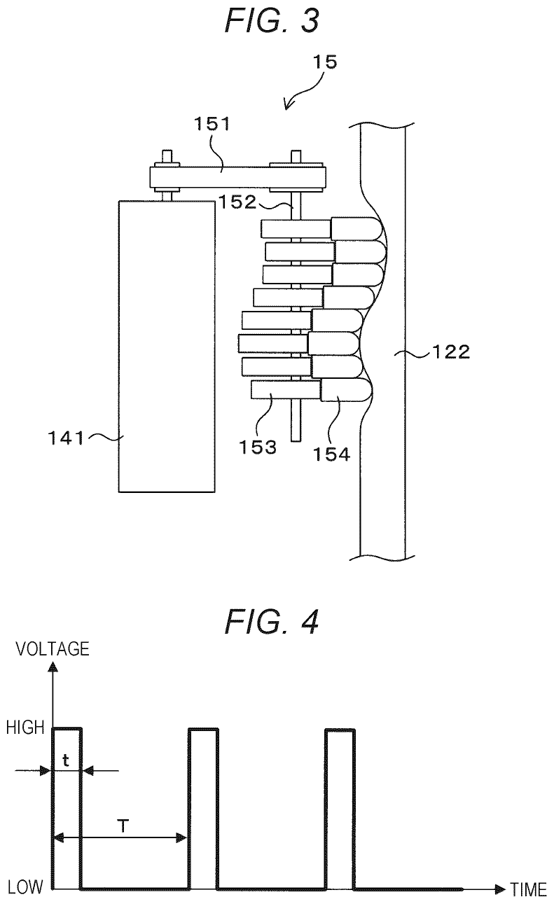

The liquid delivery unit 14 includes a motor 141, a liquid delivery mechanism 15, a motor driver 142, and a Hall sensor 143. The liquid delivery mechanism 15 is a mechanism for delivering a medicinal solution through the tube 122 by applying force to the tube 122. FIG. 3 is a schematic view illustrating a configuration example of the liquid delivery mechanism 15. A speed reducer 151 is connected to a rotation shaft of the motor 141, and a camshaft 152 is connected to the speed reducer 151. The speed reducer 151 includes a gear, a belt, and the like. A plurality of cams 153 are coupled to the camshaft 152. The plurality of cams 153 are in contact with a plurality of fingers 154. The plurality of fingers are aligned along a longitudinal direction of the tube 122. Rotation of the motor 141 is transmitted to the camshaft 152 via the speed reducer 151, the camshaft 152 rotates, the cams 153 rotate according to the rotation of the camshaft 152, the cams 153 press the fingers 154, and the fingers 154 press the tube 122. Distances by which the respective cams 153 press the fingers 154 are different from each other and change in accordance with rotation. As a result, the plurality of fingers 154 sequentially press the tube 122 from an upstream side to a downstream side of the tube 122, the tube 122 performs peristaltic movement, and the medicinal solution flows inside the tube 122 from the upstream side to the downstream side. In this manner, the medicinal solution is delivered through the tube 122.

The motor driver 142 operates the motor 141. The control unit 13 outputs a control signal for operating the motor 141 to the motor driver 142. The motor driver 142 applies a voltage to the motor 141 in accordance with the control signal from the control unit 13. In the motor 141, a rotor rotates by a voltage being applied. In this manner, the motor 141 operates.

The Hall sensor 143 is a sensor that includes a Hall element and detects a rotational position of the motor 141. The Hall sensor 143 outputs a position signal corresponding to the rotational position of the rotor of the motor 141, that is, the rotational position of the motor 141, to the control unit 13. The control unit 13 specifies rotation speed of the motor 141 based on the position signal from the Hall sensor 143 and controls operation of the motor 141.

The operation unit 111 accepts operation from the user. For example, the operation unit 111 includes a push button or a touch panel. For example, the user inputs an instruction to administer the medicinal solution by operating the operation unit 111. In accordance with the input instruction, the control unit 13 operates the motor 141, and the liquid delivery mechanism 15 operates to deliver the medicinal solution. In this manner, the infusion pump 1 performs infusion, and the medicinal solution is administered into the body of the patient 3.

The display unit 112 displays information. The display unit 112 is, for example, a liquid crystal display or an electroluminescent display (EL display). For example, the display unit 112 displays a dose or a flow rate of the medicinal solution. The sound generation unit 113 outputs sound. For example, the sound generation unit 113 comprises a speaker. The infusion pump 1 may further include a light emitting unit as an operation indicator that displays a state. The light emitting unit includes a light-emitting diode (LED) and is capable of giving a notification of abnormality of the infusion pump 1 according to a difference in light emission color of the LED.

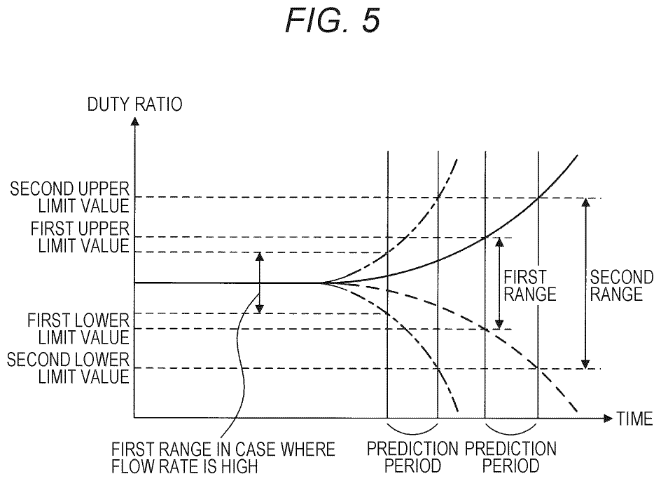

FIG. 4 is a graph indicating temporal change of a voltage to be applied to the motor 141. A high voltage or a low voltage is applied to the motor 141. The voltage to be applied to the motor 141 changes periodically. A length of one cycle of the voltage change is defined as T, and a length of a duration during which a state in which the voltage is high continues is defined as t. A duty ratio, which is a ratio of the duration during which the voltage is high in one cycle of the voltage change, is represented by t/T. In a case in which the duty ratio increases, an effective voltage to be applied to the motor 141 increases, a maximum torque of the motor 141 increases, and the rotation speed increases. The control unit 13 controls the duty ratio of the voltage to be applied to the motor 141. For example, the control unit 13 inputs a control signal indicating the duty ratio to the motor driver 142, and the motor driver 142 applies the voltage to the motor 141 at the duty ratio in accordance with the control signal.

The control unit 13 controls the motor 141 so that the flow rate of the medicinal solution becomes constant. Here, the flow rate is an amount of the medicinal solution flowing during a certain period of time. In practice, the control unit 13 performs control while changing the duty ratio of the voltage. In a case in which the load of the motor 141 is constant, an average of the duty ratios within a predetermined period is kept substantially constant. In a case in which the load increases for some reason, the rotation speed of the motor 141 decreases, and the flow rate decreases. The control unit 13 detects decrease in the rotation speed based on the position signal from the Hall sensor 143 and performs control to increase the duty ratio of the voltage. As the duty ratio of the voltage increases, the rotation speed of the motor 141 increases, the flow rate returns to the original value, and the flow rate is maintained. Conversely, in a case in which the load decreases, the control unit 13 decreases the duty ratio, so that the flow rate is maintained.

The infusion pump 1 gradually deteriorates with continuous use, which leads to occurrence of a failure. For example, contact portions between the cams 153 and the fingers 154 wear, wear powder is clogged between the plurality of fingers 154 or the speed reducer 151, the load of the motor 141 increases, and the liquid delivery mechanism 15 finally stops moving. For example, the fingers 154 wear, a pressure of pressing the tube 122 decreases, the load of the motor 141 decreases, a deviation occurs between the set flow rate and the actual flow rate of the medicinal solution, and the liquid cannot be delivered finally. When the load of the motor 141 that is controlled so that the flow rate of the medicinal solution becomes constant is out of the normal range, it is assumed that a failure occurs in the infusion pump 1. The duty ratio of the voltage to be applied to the motor 141 changes according to the load of the motor 141, and thus, the duty ratio increases in a case in which the load of the motor 141 increases, and the duty ratio decreases in a case in which the load of the motor 141 decreases. Thus, occurrence of a failure can be detected based on the duty ratio.

FIG. 5 is a graph schematically indicating an example of temporal change of the duty ratio. FIG. 5 indicates time on a horizontal axis and indicates the duty ratio of the voltage to be applied to the motor 141 on a vertical axis. The temporal change of the duty ratio in a case in which the load of the motor 141 increases is indicated by a solid line, and the temporal change of the duty ratio in a case in which the load decreases is indicated by a broken line. In practice, the duty ratio fluctuates finely up and down, and thus, FIG. 5 indicates rough tendency of the temporal change of the duty ratio. A range of the duty ratio in a state in which the load of the motor 141 is included in the normal range is defined as a second range. In a case in which the duty ratio exceeds a predetermined second upper limit value that is an upper limit of the second range, the load of the motor 141 is out of the normal range, and the infusion pump 1 is in a state in which a failure has occurred. In addition, also in a case in which the duty ratio falls below a predetermined second lower limit value that is a lower limit of the second range, the load of the motor 141 is out of the normal range, and the infusion pump 1 is in a state in which a failure has occurred. As described above, the state in which the duty ratio is out of the second range is a state in which a failure has occurred in the infusion pump 1.

In a case in which the load of the motor 141 increases and a failure occurs, as indicated in FIG. 5, the duty ratio gradually increases with the lapse of time and finally exceeds the second upper limit value. Thus, if it is detected that the duty ratio gradually increases and approaches the second upper limit value, it is possible to determine that a failure will occur in the near future, that is, there is a risk of occurrence of a failure before the failure occurs. Similarly, in a case in which the load of the motor 141 decreases and a failure occurs, the duty ratio gradually decreases with the lapse of time and finally falls below the second lower limit value. Thus, if it is detected that the duty ratio gradually decreases and approaches the second lower limit value, it is possible to determine that there is a risk of occurrence of a failure before the failure occurs.

In the present embodiment, in a case in which the duty ratio exceeds a specific first upper limit value that is smaller than the second upper limit value, it is determined that there is a risk of occurrence of a failure. The first upper limit value is a duty ratio at a point in time a predetermined period before a point in time at which the duty ratio that is gradually increasing exceeds the second upper limit value. Hereinafter, this predetermined period will be referred to as a prediction period. It is predicted that a failure will occur after lapse of the prediction period since the duty ratio has exceeded the first upper limit value. In addition, in a case in which the duty ratio falls below a specific first lower limit value that is smaller than the first upper limit value and greater than the second lower limit value, it is determined that there is a risk of occurrence of a failure. The first lower limit value is a duty ratio at a point in time the prediction period before a point in time at which the duty ratio that is gradually decreasing falls below the second lower limit value. A range of the duty ratio having the first upper limit value as an upper limit and the first lower limit value as a lower limit is defined as a first range. The first range is included in the second range.

In a case in which the flow rate of the medicinal solution to be delivered by the infusion pump 1 is high, deterioration is accelerated, the temporal change of the load of the motor 141 is large, and the temporal change of the duty ratio of the voltage to be applied to the motor 141 is also large. For example, the higher the flow rate, the more easily the contact portions between the cams 153 and the fingers 154 wear, the load of the motor 141 increases in a shorter period of time, and the duty ratio increases in a shorter period of time. For example, the higher the flow rate, the more easily the fingers 154 wear, the load of the motor 141 decreases in a shorter period of time, and the duty ratio decreases in a shorter period of time. In FIG. 5, the temporal change of the duty ratio in a case in which the flow rate is high is indicated by an alternate long and short dash line.

In a case in which the flow rate is high, the temporal change of the duty ratio is large, and thus, the duty ratio at the point in time the prediction period before the point in time at which the duty ratio exceeds the second upper limit value becomes a lower value. In other words, the first upper limit value becomes lower. Similarly, the duty ratio at the point in time the prediction period before the point in time at which the duty ratio falls below the second lower limit becomes a higher value. In other words, the first lower limit value becomes higher. As described above, as the flow rate is higher, the first upper limit value becomes lower, the first lower limit value becomes higher, and the first range becomes narrower.

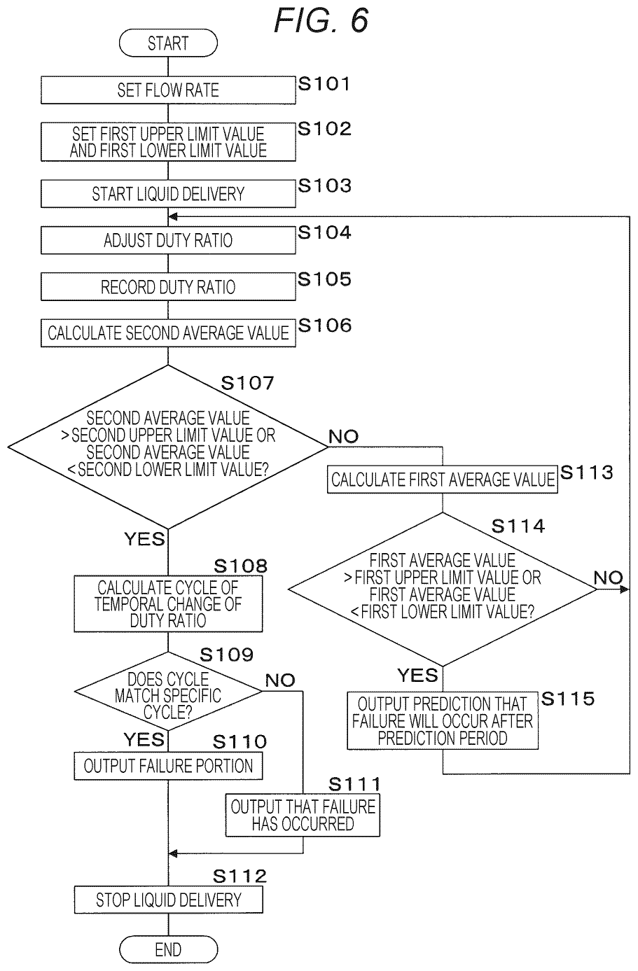

In the present embodiment, the infusion pump 1 executes an information processing method for determining whether or not a failure has occurred at the present time and determining whether or not there is a risk of occurrence of a failure in the near future. FIG. 6 is a flowchart indicating an example of procedure of processing to be executed by the infusion pump 1 according to the first embodiment. Hereinafter, step will be abbreviated as S. The control unit 13 executes the following processing by the calculation unit 131 executing information processing in accordance with the computer program 133.

The control unit 13 sets the flow rate of the medicinal solution to be delivered by the infusion pump 1 (S101). For example, the infusion pump 1 accepts an instruction to set the flow rate of the medicinal solution by the user operating the operation unit 111, and the control unit 13 sets the flow rate of the medicinal solution. For example, the infusion pump 1 accepts an instruction to set the flow rate of the medicinal solution from an external device, and the control unit 13 sets the flow rate of the medicinal solution. The flow rate of the medicinal solution may be set in advance.

Next, the control unit 13 sets the first upper limit value and the first lower limit value according to the flow rate (S102). In S102, the control unit 13 sets the first upper limit value and the first lower limit value such that the first upper limit value becomes lower and the first lower limit value becomes higher as the flow rate is higher. In other words, the control unit 13 sets the first upper limit value and the first lower limit value such that the first range becomes narrower as the flow rate is higher. For example, the control unit 13 stores data in which the flow rate is associated with the first upper limit value and the first lower limit value in the memory 132 in advance and sets the first upper limit value and the first lower limit value to values associated with the set flow rate. For example, the control unit 13 calculates the first upper limit value and the first lower limit value according to the flow rate using a predetermined function. The first upper limit value and the first lower limit value may be set in advance according to the flow rate. By making the first range narrower as the flow rate is higher, it is possible to predict a failure of the infusion pump 1 while allowing enough time for the prediction period even in a case in which the flow rate is high and deterioration is accelerated.

The infusion pump 1 starts liquid delivery (S103). For example, the infusion pump 1 accepts an instruction to administer the medicinal solution by the user operating the operation unit 111 and starts liquid delivery. The control unit 13 outputs a control signal for operating the motor 141 to the motor driver 142, and the motor driver 142 accepts the control signal and applies a voltage to the motor 141 according to the control signal. The motor 141 rotates by the voltage being applied, the liquid delivery mechanism 15 operates by the rotation of the motor 141, and the medicinal solution is delivered through the tube 122. The delivered medicinal solution is administered into the body of the patient 3 through the infusion tube 22 and the injection needle 21. The infusion pump 1 may accept an instruction to administer the medicinal solution from an external device and start liquid delivery. The infusion pump 1 may start liquid delivery when it is time to start medication determined in advance.

The control unit 13 adjusts the duty ratio of the voltage to be applied to the motor 141 so that the flow rate of the medicinal solution becomes constant (S104). In S104, the control unit 13 specifies the rotation speed of the motor 141 based on the position signal from the Hall sensor 143 and adjusts the duty ratio based on the rotation speed. To make the flow rate of the medicinal solution constant, it is necessary to make the rotation speed of the motor 141 constant. In a case in which the specified rotation speed is lower than the rotation speed for implementing the set flow rate, the control unit 13 changes the duty ratio to be higher so as to increase the rotation speed. In a case in which the specified rotation speed is higher than the rotation speed for implementing the set flow rate, the control unit 13 changes the duty ratio to a lower value to decrease the rotation speed. The control unit 13 inputs a control signal indicating the changed duty ratio to the motor driver 142, and the motor driver 142 applies a voltage to the motor 141 at the duty ratio according to the control signal, and the rotation speed of the motor 141 is changed. In this manner, the control unit 13 adjusts the duty ratio according to fluctuation of the load of the motor 141.

The control unit 13 records the duty ratio (S105). The control unit 13 records the duty ratio by storing a value of the duty ratio in the memory 132. The duty ratio is periodically recorded, and the value of the duty ratio at each past point in time is stored in the memory 132 as a history. The processing in S105 corresponds to a recording unit.

The control unit 13 calculates a second average value obtained by averaging the duty ratios during a predetermined second period (S106). In S106, the control unit 13 averages a plurality of duty ratios during the latest second period included in the history of the duty ratios stored in the memory 132. The second period is, for example, 20 seconds.

The control unit 13 determines whether the second average value exceeds the second upper limit value or whether the second average value is less than the second lower limit value (S107). The second upper limit value and the second lower limit value are stored in the memory 132 in advance. Through the processing in S107, the control unit 13 determines whether or not a failure has occurred in the infusion pump 1. A state in which the second average value exceeds the second upper limit value or the second average value is less than the second lower limit value is a state in which a failure has occurred in the infusion pump 1. A state in which the second average value is equal to or less than the second upper limit value and equal to or greater than the second lower limit value is a state in which no failure has occurred yet. By performing the determination using the average value of the duty ratios without using the instantaneous duty ratio, it is possible to accurately determine whether or not a failure has occurred. Note that the control unit 13 may perform processing of determining a state in which the second average value is equal to or greater than the second upper limit value or the second average value is equal to or less than the second lower limit value as a state in which a failure has occurred.

In a case in which the second average value exceeds the second upper limit value or the second average value is less than the second lower limit value (S107: Yes), the control unit 13 calculates a cycle of the temporal change of the duty ratio (S108). In S108, the control unit 13 calculates the cycle of the temporal change of the duty ratio based on the latest predetermined number of duty ratios included in the history of the duty ratios stored in the memory 132. For example, the control unit 13 performs Fourier transform on the temporal change of the predetermined number of values of the duty ratios and specifies the cycle based on the result of the Fourier transform.

Next, the control unit 13 determines whether or not the calculated cycle matches a specific cycle (S109). For example, in a case in which one of the plurality of fingers 154 breaks down, the load of the motor 141 fluctuates significantly when the finger 154 that has broken down compresses the tube 122. The duty ratio changes in the same cycle as the cycle in which one finger 154 among the plurality of fingers 154 compresses the tube 122. For example, in a case in which part of a gear included in the speed reducer 151 breaks down, the duty ratio changes in a specific cycle according to a wave number of the gear, or the like. As described above, the duty ratio changes in a cycle corresponding to a place where a failure has occurred, and thus, the place where the failure has occurred can be specified based on the cycle of the duty ratio. A value of the specific cycle is stored in the memory 132 in advance.

In a case in which the cycle of the duty ratio matches the specific cycle (S109: Yes), the control unit 13 specifies a failure portion according to the specific cycle and outputs that a failure has occurred in the infusion pump 1 and the failure portion (S110). In S110, the control unit 13 displays, on the display unit 112, an image including a sentence indicating that the failure has occurred and the failure portion.

In a case in which the cycle of the duty ratio does not match the specific cycle (S109: No), the control unit 13 outputs that a failure has occurred in the infusion pump 1 (S111). In S111, the control unit 13 displays an image including a sentence indicating that the failure has occurred on the display unit 112. In S110 and S111, the control unit 13 may cause the sound generation unit 113 to generate sound indicating that the failure has occurred or may make a notification that the failure has occurred by an operation indicator.

After the processing in S110 or S111 ends, the infusion pump 1 stops liquid delivery (S112). In S112, the control unit 13 inputs a control signal for stopping the motor 141 to the motor driver 142, the motor driver 142 stops application of the voltage to the motor 141, the motor 141 is stopped, and the liquid delivery is stopped. After the processing in S112 ends, the infusion pump 1 ends the processing.

In a case in which the second average value is equal to or less than the second upper limit value and equal to or greater than the second lower limit value (S107: No), the control unit 13 calculates a first average value by averaging the duty ratios during a predetermined first period (S113). In S113, the control unit 13 averages the plurality of duty ratios during the latest first period included in the history of the duty ratios stored in the memory 132. The first period is longer than the second period. The first period is, for example, 3 minutes.

The control unit 13 determines whether the first average value exceeds the first upper limit value or whether the first average value is less than the first lower limit value (S114). Through the processing in S114, the control unit 13 determines whether or not there is a risk of occurrence of a failure in the infusion pump 1. A state in which the first average value exceeds the first upper limit value or the first average value is less than the first lower limit value is a state in which it is predicted that a failure will occur in the infusion pump 1 at the point in time at which a period equivalent to the prediction period has elapsed, and there is a high risk of occurrence of a failure in the near future. A state in which the first average value is equal to or less than the first upper limit value and equal to or greater than the first lower limit value is a state in which a risk of occurrence of a failure is low.

The first period is longer than the second period, and thus, the first average value is an average of duty ratios obtained in a longer period. For this reason, the first average value indicates tendency of the temporal change of the duty ratio in a longer period instead of an instantaneous duty ratio. It is possible to accurately determine whether or not there is a risk of occurrence of a failure by performing the determination based on the tendency of the temporal change of the duty ratio in a long period. Note that the control unit 13 may perform processing of determining that there is a risk of a failure in a state in which the first average value is equal to or greater than the first upper limit value or the first average value is equal to or less than the first lower limit value. The processing in S114 corresponds to a determination unit.

In a case in which the first average value is equal to or less than the first upper limit value and equal to or greater than the first lower limit value (S114: No), the control unit 13 determines that there is no risk of occurrence of a failure in the infusion pump 1 and returns the processing to S104. In a case in which the first average value exceeds the first upper limit value or the first average value is less than the first lower limit value (S114: Yes), the control unit 13 determines that there is a risk of occurrence of a failure in the infusion pump 1 and outputs prediction that a failure will occur in the infusion pump 1 after the prediction period (S115). The processing in S115 corresponds to an output unit. After the processing in S115 ends, the control unit 13 returns the processing to S104. In S115, the control unit 13 displays an image including a sentence indicating that it is predicted that a failure will occur in the infusion pump 1 after the prediction period on the display unit 112.

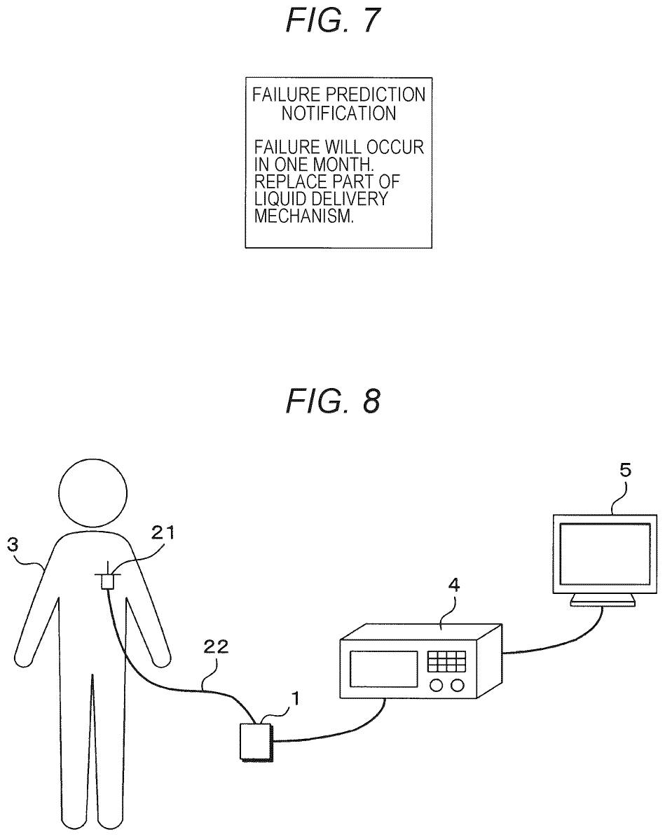

FIG. 7 is a schematic view illustrating an output example of prediction of occurrence of a failure. An image indicating that it is predicted that a failure will occur in the infusion pump 1 after the prediction period is displayed. FIG. 7 illustrates an example in which the prediction period is one month. In addition, FIG. 7 illustrates an example in which a proposal for replacing a part of the liquid delivery mechanism 15 is output. In a case in which a deteriorated part is replaced in response to output of a proposal for replacing a part, occurrence of a failure is avoided in advance. In S115, the control unit 13 may cause the sound generation unit 113 to generate sound indicating prediction that a failure will occur in the infusion pump 1.

In S115, the control unit 13 may display, on the display unit 112, an image including a sentence indicating that it is predicted that a failure will occur with a specific color. For example, the control unit 13 displays an image in which a base color of the sentence is set to a specific color on the display unit 112. For example, the control unit 13 may display an image including a sentence indicating that the failure has occurred on the display unit 112 using red in S110 and S111 and may display an image including a sentence indicating that it is predicted that the failure will occur on the display unit 112 using yellow in S115. By adding a specific color to the image, attention of the user is attracted, and the user can easily recognize a risk of occurrence of a failure.

The processing in S104 to S115 is repeatedly executed, and the medicinal solution is administered. In a case in which a dose of the medicinal solution reaches a predetermined set value or in a case in which the infusion pump 1 accepts an instruction to end administration, the infusion pump 1 stops liquid delivery and ends the processing. The infusion pump 1 accepts an instruction to end administration, for example, by the user operating the operation unit 111 or by receiving an instruction from an external device.

In the above description, the processing of comparing the average value of the duty ratio with the specific upper limit value and lower limit value has been described, but the infusion pump 1 may perform processing of comparing a value obtained by processing the average value with the specific upper limit value and lower limit value. For example, in S107, the control unit 13 compares a value obtained by dividing the second average value by an initial value of the second average value with the second upper limit value and the second lower limit value. In this form, the second range is a range obtained by multiplying a range determined by the second upper limit value and the second lower limit value by the initial value of the second average value. For example, in S114, the control unit 13 compares a value obtained by dividing the first average value by an initial value of the first average value with the first upper limit value and the first lower limit value. In this form, the first range is a range obtained by multiplying a range determined by the first upper limit value and the first lower limit value by the initial value of the first average value. In these forms, it is possible to accurately determine whether or not a failure has occurred and whether or not there is a risk of occurrence of a failure based on change in the average value of the duty ratio. The infusion pump 1 may use a value obtained by dividing the average value of the duty ratio by other values.

Furthermore, in the above description, the processing of setting the first upper limit value and the first lower limit value according to the flow rate has been described, but the infusion pump 1 may set the first upper limit value and the first lower limit value according to a temperature. FIG. 5 indicates that the temporal change of the duty ratio of the voltage to be applied to the motor 141 varies depending on the flow rate, but similarly, the temporal change of the duty ratio varies also in a case in which the temperature varies. In a case in which the temperature is high, the temporal change of the load of the motor 141 is large and the temporal change of the duty ratio is large due to a cause such as rapid deterioration of a lubricant used in the liquid delivery mechanism 15. For example, the higher the temperature, the more easily the contact portions between the cams 153 and the fingers 154 wear, the load of the motor 141 increases in a shorter period of time, and the duty ratio increases in a shorter period of time.

For example, the infusion pump 1 includes a temperature sensor and performs processing of setting the first upper limit value and the first lower limit value according to the measured temperature. The control unit 13 sets the first upper limit value and the first lower limit value such that the first upper limit value becomes lower and the first lower limit value becomes higher as the temperature is higher. For example, the control unit 13 stores data in which the temperature is associated with the first upper limit value and the first lower limit value in the memory 132 in advance and sets the first upper limit value and the first lower limit value to values associated with the measured temperature. The control unit 13 calculates the first upper limit value and the first lower limit value according to the temperature using a predetermined function. The infusion pump 1 may set the first upper limit value and the first lower limit value according to both the flow rate and the temperature.

As described above in detail, in the present embodiment, the infusion pump 1 performs liquid delivery while maintaining a constant flow rate and determines whether or not there is a risk of occurrence of a failure based on the duty ratio of the voltage to be applied to the motor 141. In the infusion pump 1, the load of the motor 141 increases or decreases with deterioration, and the duty ratio changes. In a case in which the temporal change of the duty ratio is the same as temporal change of the duty ratio immediately before a failure occurs in the infusion pump 1, it can be predicted that a failure will occur in the near future. It is therefore possible to determine whether or not there is a risk of occurrence of a failure based on the history of the duty ratio. In other words, it is possible to predict occurrence of a failure in the infusion pump 1 before the failure occurs.

In the present embodiment, in a case in which there is a risk of occurrence of a failure in the infusion pump 1, prediction of occurrence of a failure is output. The user can easily recognize a risk of occurrence of a failure. A failure can be predicted before the failure occurs, so that the deteriorated part can be replaced before the failure occurs. In addition, it is also possible to avoid use of the infusion pump 1 in which a failure may occur. In this manner, occurrence of a failure in the infusion pump 1 can be avoided in advance. Even if the user is not skilled in handling the infusion pump 1, it is possible to take appropriate measures such as replacement of a part without overlooking a sign of a failure. Compared to the related art, maintenance of the infusion pump 1 becomes efficient, and the burden on the user is reduced. Occurrence of a failure in the infusion pump 1 can be avoided, so that an adverse effect on the patient 3 due to a failure in the infusion pump 1 is avoided, and safety of the treatment is improved.

### Second Embodiment

In a second embodiment, a form in which a failure in the infusion pump 1 is predicted outside the infusion pump 1 will be described. FIG. 8 is a schematic view illustrating a configuration example of an information processing system according to the second embodiment. In a similar manner to the first embodiment, the infusion pump 1 administers a medicinal solution into the body of the patient 3. The infusion pump 1 is connected to the information processing apparatus 4 via a communication line. The information processing apparatus 4 predicts a failure in the infusion pump 1 and outputs information regarding the infusion pump 1. For example, the information processing apparatus 4 is disposed in a room of the patient 3, and a user such as the patient 3 or a medical worker confirms information regarding the infusion pump 1 using the information processing apparatus 4. A display device 5 is connected to the information processing apparatus 4 via a communication line. The display device 5 is, for example, a liquid crystal display or an EL display. The information processing apparatus 4 displays information on the infusion pump 1 on the display device 5. For example, the display device 5 is disposed at a place away from the room of the patient 3, and a user such as a medical worker or a relative of the patient checks information on the infusion pump 1 using the display device 5 from a position away from the patient 3.

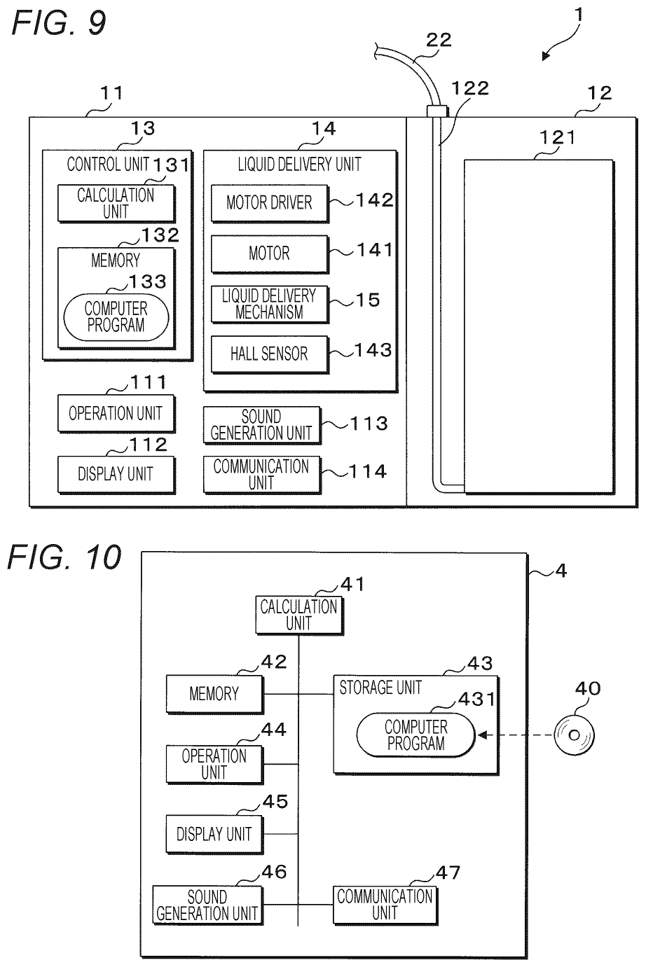

FIG. 9 is a block diagram illustrating an example of an internal functional configuration of the infusion pump 1 according to the second embodiment. The infusion pump 1 includes a communication unit 114. The configurations of other parts of the infusion pump 1 are similar to those of the first embodiment. The communication unit 114 communicates with the information processing apparatus 4 via a communication line. The communication unit 114 may communicate with the information processing apparatus 4 using wireless communication.

FIG. 10 is a block diagram illustrating an internal configuration example of the information processing apparatus 4 according to the second embodiment. The information processing apparatus 4 includes a calculation unit 41, a memory 42, a storage unit 43, an operation unit 44, a display unit 45, a sound generation unit 46, and a communication unit 47. The calculation unit 41 comprises, for example, a CPU, a GPU, or a multi-core CPU. The calculation unit 41 may comprise a quantum computer. The memory 42 stores temporary data generated in association with calculation. The memory 42 is, for example, a RAM. The storage unit 43 is non-volatile and is, for example, a hard disk or a non-volatile semiconductor memory.

The storage unit 43 stores a computer program 431. For example, at the time of manufacturing the information processing apparatus 4, the computer program 431 is read from the recording medium 40 such as an optical disk or a portable memory that stores the computer program 431 and is stored in the storage unit 43. The computer program 431 may be downloaded using the communication unit 47 and stored in the storage unit 43. The calculation unit 41 executes necessary processing according to the computer program 431.

The operation unit 44 accepts operation from the user. For example, the operation unit 44 includes a push button or a touch panel. For example, the user performs various kinds of settings by operating the operation unit 44. The display unit 45 displays information. The display unit 45 is, for example, a liquid crystal display or an EL display. The sound generation unit 46 outputs sound. For example, the sound generation unit 46 comprises a speaker.

The communication unit 47 is connected to the infusion pump 1 and the display device 5 via a communication line. The communication unit 47 may use wireless communication. The information processing apparatus 4 acquires information from the infusion pump 1 using the communication unit 47 and causes the display device 5 to display the information. For example, the information processing apparatus 4 displays information similar to the information displayed on the display unit 45 on the display device 5. The information processing apparatus 4 may be connected to a plurality of infusion pumps 1 and predict a failure in the plurality of infusion pumps 1. The information processing apparatus 4 may be connected to a plurality of display devices 5 and display information on the plurality of display devices 5.

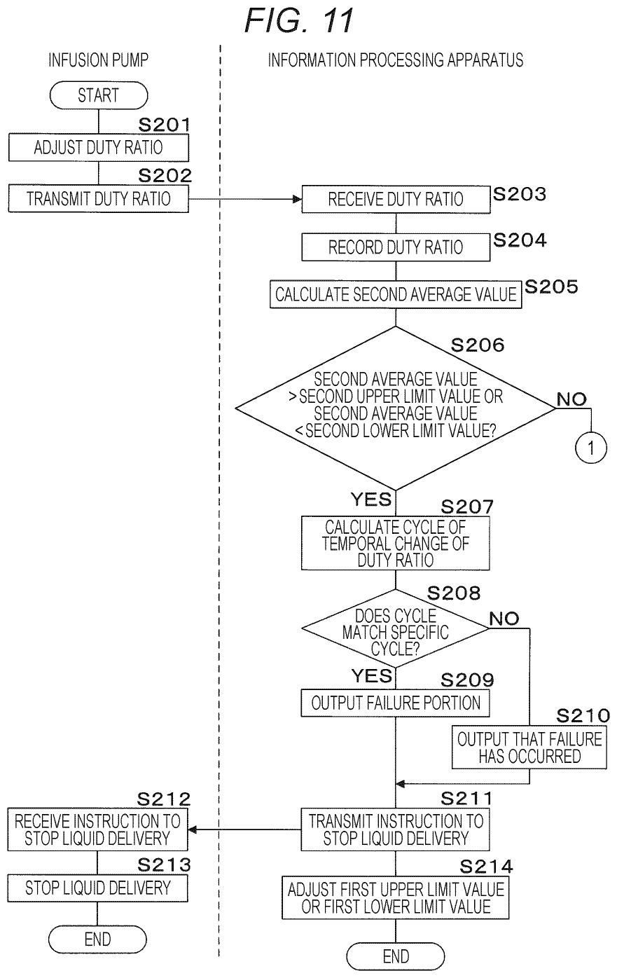

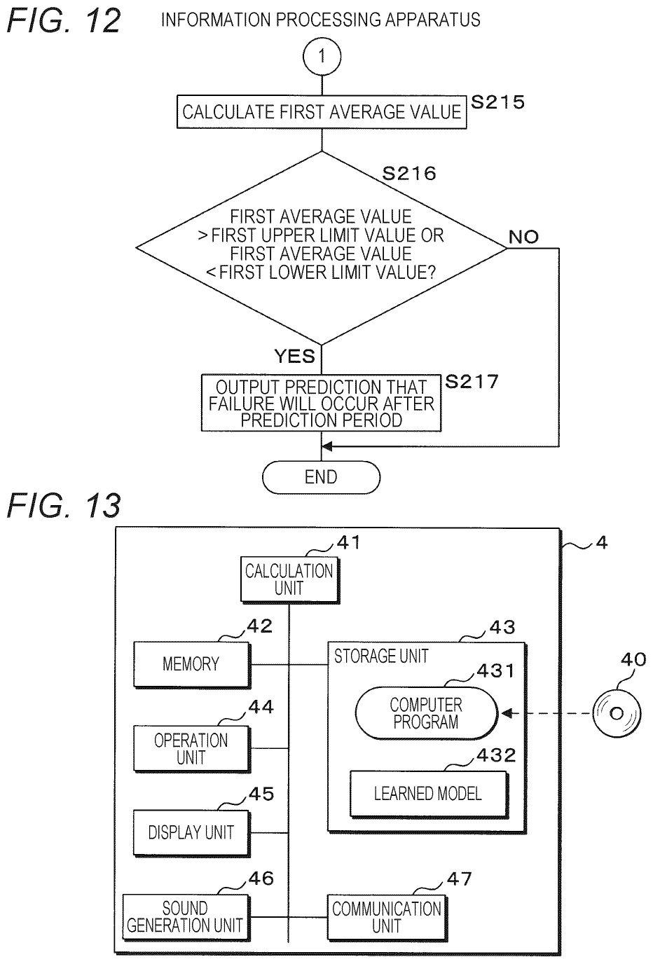

FIGS. 11 and 12 are flowcharts indicating an example of procedure of processing to be executed by the information processing system according to the second embodiment. The calculation unit 41 executes information processing according to the computer program 431. The infusion pump 1 performs liquid delivery similarly to that performed in the first embodiment, and the medicinal solution is administered to the patient 3. A flow rate of the medicinal solution is set in a similar manner to that in the first embodiment. In a similar manner to the first embodiment, the control unit 13 adjusts the duty ratio of the voltage to be applied to the motor 141 so that the flow rate of the medicinal solution becomes constant (S201).

The infusion pump 1 transmits the duty ratio to the information processing apparatus 4 (S202). In S202, the control unit 13 causes the communication unit 114 to transmit the adjusted value of the duty ratio to the information processing apparatus 4. The information processing apparatus 4 receives the duty ratio of the voltage to be applied to the motor 141 of the infusion pump 1 by receiving the value of the duty ratio transmitted from the infusion pump 1 by the communication unit 47 (S203). Through the processing in S203, the information processing apparatus 4 acquires the duty ratio from the infusion pump 1. The processing in S203 corresponds to an acquisition unit. The calculation unit 41 and the communication unit 47 function as the acquisition unit by performing the processing in S203 of acquiring the duty ratio by communication.

The information processing apparatus 4 records the received duty ratio (S204). In S204, the calculation unit 41 records the duty ratio by storing the value of the duty ratio in the storage unit 43. The duty ratio is sequentially recorded, and the value of the duty ratio at each point in time in the past is stored in the storage unit 43 as a history. The processing in S204 corresponds to a recording unit.

The information processing apparatus 4 calculates a second average value by averaging duty ratios during the predetermined second period (S205). In S205, the calculation unit 41 averages the plurality of duty ratios during the latest second period included in the history of the duty ratios stored in the storage unit 43. Next, the information processing apparatus 4 determines whether the second average value exceeds the second upper limit value or whether the second average value is less than the second lower limit value (S206). The second upper limit value and the second lower limit value are stored in the storage unit 43 in advance. Through the processing in S206, the calculation unit 41 determines whether or not a failure has occurred in the infusion pump 1.

In a case in which the second average value exceeds the second upper limit value or the second average value is less than the second lower limit value (S206: Yes), the information processing apparatus 4 calculates a cycle of the temporal change of the duty ratio (S207). In S207, the calculation unit 41 calculates the cycle of the temporal change of the duty ratio based on the latest predetermined number of duty ratios included in the history of the duty ratio stored in the storage unit 43.

Next, the calculation unit 41 determines whether or not the calculated cycle matches the specific cycle (S208). The value of the specific cycle is stored in the storage unit 43 in advance. In a case in which the cycle of the duty ratio matches the specific cycle (S208: Yes), the information processing apparatus 4 specifies a failure portion according to the specific cycle and outputs that a failure has occurred in the infusion pump 1 and the failure portion (S209). In S209, the calculation unit 41 displays, on the display unit 45, an image including a sentence indicating that the failure has occurred and the failure portion. In addition, the calculation unit 41 causes the communication unit 47 to transmit data indicating that the failure has occurred and the failure portion to the display device 5. The display device 5 displays an image including a sentence indicating that the failure has occurred and the failure portion based on the data from the information processing apparatus 4.

In a case in which the cycle of the duty ratio does not match the specific cycle (S208: No), the information processing apparatus 4 outputs that a failure has occurred in the infusion pump 1 (S210). In S210, the calculation unit 41 displays an image including a sentence indicating that the failure has occurred on the display unit 45. In addition, the calculation unit 41 causes the communication unit 47 to transmit data indicating that the failure has occurred to the display device 5. The display device 5 displays an image including a sentence indicating that the failure has occurred based on the data from the information processing apparatus 4. In S209 and S210, the calculation unit 41 may cause the sound generation unit 46 to generate sound indicating that the failure has occurred.

After the processing in S209 or S210 ends, the information processing apparatus 4 transmits an instruction to stop liquid delivery to the infusion pump 1 (S211). In S211, the calculation unit 41 causes the communication unit 47 to transmit the instruction to stop liquid delivery to the infusion pump 1. The infusion pump 1 receives the instruction to stop liquid delivery by the communication unit 114 (S212), stops liquid delivery (S213), and ends the processing. In S213, the control unit 13 inputs a control signal for stopping the motor 141 to the motor driver 142, the motor 141 is stopped, and the liquid delivery is stopped.

After the processing in S211 ends, the information processing apparatus 4 adjusts the first upper limit value or the first lower limit value according to the history of the duty ratio (S214). For example, the calculation unit 41 specifies the duty ratio at a point in time the prediction period before a point in time at which the second average value exceeds the second upper limit value based on the history of the duty ratio stored in the storage unit 43. The calculation unit 41 calculates an average value by averaging duty ratios during the first period including the specified duty ratio and duty ratios before and after the point in time of the specified duty ratio and sets the obtained average value as the first upper limit value. For example, the calculation unit 41 specifies the duty ratio at a point in time the prediction period before a point in time at which the second average value falls below the second lower limit value, and sets an average value obtained by averaging the duty ratios during the first period including the specified duty ratio and the duty ratios before and after the point in time of the specified duty ratio as the first lower limit value.

The calculation unit 41 may calculate a new first upper limit value or first lower limit value by calculating an average of the calculated average value of the duty ratios and the past first upper limit value or first lower limit value. The storage unit 43 stores the first upper limit value and the first lower limit value for other infusion pumps, and the calculation unit 41 may calculate a new first upper limit value or first lower limit value by calculating an average of the calculated average value of the duty ratios and the first upper limit value or first lower limit value for other infusion pumps. The calculation unit 41 may set the first upper limit value or the first lower limit value for each flow rate or each temperature of the medicinal solution to be delivered by the infusion pump 1. The calculation unit 41 may adjust both the first upper limit value and the first lower limit value.

The first upper limit value or the first lower limit value can be adjusted to a value in accordance with an actual situation based on the history of the duty ratio in the infusion pump 1 in which a failure has actually occurred. When it is determined whether or not there is a risk of occurrence of a failure in other infusion pumps 1, it is possible to more accurately determine whether or not there is a risk by using the adjusted first upper limit value or first lower limit value. The calculation unit 41 stores the adjusted first upper limit value or first lower limit value in the storage unit 43 and ends the processing.

In a case in which the second average value is equal to or less than the second upper limit value and equal to or greater than the second lower limit value (S206: No), the information processing apparatus 4 calculates a first average value by averaging the duty ratios during the predetermined first period (S215). In S215, the calculation unit 41 averages the plurality of duty ratios during the latest first period included in the history of the duty ratios stored in the storage unit 43. The information processing apparatus 4 determines whether the first average value exceeds the first upper limit value or whether the first average value is less than the first lower limit value (S216). Through the processing in S216, the control unit 13 determines whether or not there is a risk of occurrence of a failure in the infusion pump 1.

In S216, the calculation unit 41 performs determination using the first upper limit value or the first lower limit value stored in the storage unit 43. The first upper limit value and the first lower limit value to be used are values adjusted in S214. By adjusting the duty ratio based on the history of the duty ratio in the infusion pump 1 in which the failure has actually occurred, the first upper limit value and the first lower limit value in accordance with an actual situation can be obtained. By using such a first upper limit value and first lower limit value, it is possible to accurately determine whether or not there is a risk of occurrence of a failure in the infusion pump 1. In a case in which the first upper limit value or the first lower limit value is not set, the information processing apparatus 4 may set the first upper limit value or the first lower limit value based on the flow rate or the temperature of the medicinal solution to be delivered by the infusion pump 1 in a similar manner to the first embodiment.

In a case in which the first average value is equal to or less than the first upper limit value and equal to or greater than the first lower limit value (S216: No), the information processing apparatus 4 ends the processing. In a case in which the first average value exceeds the first upper limit value or the first average value is less than the first lower limit value (S216: Yes), the information processing apparatus 4 determines that there is a risk of occurrence of a failure in the infusion pump 1 and outputs prediction that the failure will occur in the infusion pump 1 after the prediction period (S217). In S217, the calculation unit 41 displays an image including a sentence indicating that it is predicted that the failure will occur in the infusion pump 1 after the prediction period on the display unit 45.

In addition, the calculation unit 41 causes the communication unit 47 to transmit data indicating that it is predicted that the failure will occur to the display device 5. The display device 5 displays an image indicating that it is predicted that the failure will occur based on the data from the information processing apparatus 4. For example, an image similar to the image illustrated in FIG. 7 is displayed on the display unit 45 and the display device 5. The calculation unit 41 may generate sound indicating prediction that the failure will occur in the infusion pump 1 from the sound generation unit 46. Alternatively, the calculation unit 41 may cause the communication unit 47 to transmit data indicating that it is predicted that the failure will occur to the infusion pump 1, and the infusion pump 1 may output the prediction that the failure will occur by the display unit 112, the operation indicator, or the sound generation unit 113. The processing in S217 corresponds to an output unit.

In S217, the information processing apparatus 4 may add a specific color and display an image including a sentence indicating that it is predicted that the failure will occur on the display unit 45 or the display device 5. For example, the information processing apparatus 4 displays an image in which a base color of the sentence is set to a specific color on the display unit 45 or the display device 5. For example, the information processing apparatus 4 may display an image including a sentence indicating that the failure has occurred on the display unit 45 or the display device 5 using red in S209 and S210, and may display an image including a sentence indicating that it is predicted that the failure will occur on the display unit 45 or the display device 5 using yellow in S217. By applying a specific color to the image, attention of the user is attracted.

After the processing in S217 ends, the information processing apparatus 4 ends the processing. While the infusion pump 1 is operating, the processing from S201 to S217 is repeatedly executed. In a case in which a plurality of infusion pumps 1 are connected to the information processing apparatus 4, the information processing system executes processing for predicting a failure of the plurality of infusion pumps 1.

Although the processing of comparing the average value of the duty ratio with the specific upper limit value and the specific lower limit value has been described above, the information processing apparatus 4 may perform processing of comparing a value obtained by processing the average value with the specific upper limit value and the specific lower limit value. For example, in S206, the calculation unit 41 compares a value obtained by dividing the second average value by an initial value of the second average value with the second upper limit value and the second lower limit value. For example, in S216, the calculation unit 41 compares a value obtained by dividing the first average value by an initial value of the first average value with the first upper limit value and the first lower limit value. For example, in S214, the calculation unit 41 specifies the duty ratio at a point in time the prediction period before a point in time at which the value obtained by dividing the second average value by the initial value of the second average value exceeds the second upper limit value and sets a value obtained by dividing the average of the duty ratios during the first period including the specified duty ratio by the initial value of the first average value as the first upper limit value. For example, the calculation unit 41 specifies the duty ratio at a point in time the prediction period before a point in time at which the value obtained by dividing the second average value by the initial value of the second average value falls below the second lower limit value and sets a value obtained by dividing the average of the duty ratios during the first period including the specified duty ratio by the initial value of the first average value as the first lower limit value. The information processing apparatus 4 may use a value obtained by dividing the average value of the duty ratio by other values. Furthermore, the information processing apparatus 4 may perform processing of adjusting and resetting the second lower limit value and the second upper limit value according to a fluctuation amount from the initial value of the duty ratio.

In the second embodiment, the form in which the information processing apparatus 4 is a dedicated device has been described, but the information processing apparatus 4 may be a general-purpose computer such as a personal computer. Alternatively, the information processing apparatus 4 may execute part or all of the processing using a cloud. The information processing system does not have to include the display device 5. The information processing apparatus 4 may output necessity to stop liquid delivery using the display unit 45 or the sound generation unit 46 without transmitting the instruction to stop liquid delivery to the infusion pump 1 in S211, and the user may perform operation of stopping liquid delivery on the infusion pump 1. Alternatively, the information processing apparatus 4 may transmit the instruction to stop liquid delivery to the infusion pump 1 in S211, and the infusion pump 1 may make a notification of necessity to stop liquid delivery using the display unit 112, the sound generation unit 113, or the operation indicator, and the user may perform operation of stopping liquid delivery on the infusion pump 1.

As described above in detail, the infusion pump 1 delivers liquid while maintaining a constant flow rate, and the information processing apparatus 4 determines whether or not there is a risk of occurrence of a failure based on the duty ratio of the voltage to be applied to the motor 141 of the infusion pump 1. Also in the second embodiment, it is possible to predict occurrence of a failure in the infusion pump 1 before the failure occurs, and the user can easily recognize a risk of occurrence of a failure. By the information processing apparatus 4 outputting prediction of occurrence of a failure, many people such as a medical worker can easily recognize a risk of occurrence of a failure in the infusion pump 1. Also in the second embodiment, maintenance of the infusion pump 1 becomes more efficient than the related art. In addition, it is possible to avoid occurrence of a failure in the infusion pump 1, and safety of treatment is improved.

### Third Embodiment

In a third embodiment, a mode in which a failure of the infusion pump 1 is predicted using a learned model will be described. In the third embodiment, in a similar manner to the second embodiment, a failure of the infusion pump 1 is predicted by the information processing system. FIG. 13 is a block diagram illustrating an internal configuration example of the information processing apparatus 4 according to the third embodiment. The information processing apparatus 4 includes a learned model 432 to be used to predict a failure of the infusion pump 1. The learned model 432 is implemented by the calculation unit 41 executing information processing according to a computer program 431. The storage unit 43 stores data necessary for implementing the learned model 432.

The learned model 432 may comprise hardware. For example, the learned model 432 may comprise hardware including a processor and a memory that stores necessary programs and data. Alternatively, the learned model 432 may be implemented using a quantum computer. Alternatively, the learned model 432 may be provided outside the information processing apparatus 4, and the information processing apparatus 4 may execute processing using the external learned model 432. For example, the learned model 432 may be implemented using a cloud. Configurations and functions of other portions of the information processing apparatus 4 are similar to those of the second embodiment. In addition, configurations of portions of the information processing system other than the information processing apparatus 4 are similar to those of the second embodiment.

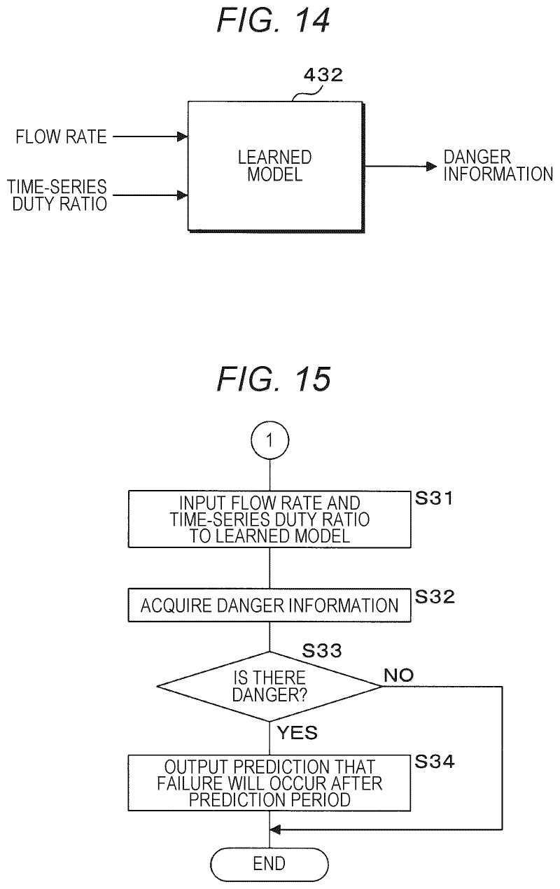

FIG. 14 is a conceptual diagram illustrating functions of the learned model 432. A flow rate of the medicinal solution to be delivered by the infusion pump 1 and a time-series duty ratio of the voltage to be applied to the motor 141 of the infusion pump 1 are input to the learned model 432. The learned model 432 is learned in advance so as to output danger information regarding a risk of occurrence of a failure in the infusion pump 1 in a case in which the flow rate and the time-series duty ratio are input. For example, the danger information indicates whether or not there is a risk of occurrence of a failure in the infusion pump 1 after the prediction period. Alternatively, the danger information indicates a probability of occurrence of a failure in the infusion pump 1 after the prediction period. For example, the learned model 432 comprises a neural network such as a convolutional neural network (CNN), a long short-term memory (LSTM), or a transformer. The learned model 432 may be a model using a method other than the neural network.

The learned model 432 is generated by machine learning using training data including a time-series duty ratio obtained in the past and a flow rate when the time-series duty ratio is obtained. The learned model 432 is learned by a learning device using a computer. For example, the training data includes a plurality of time-series duty ratios including a duty ratio at a point in time the prediction period before the point in time at which a failure has occurred in the infusion pump 1 and duty ratios before and after the point in time at which the failure has occurred. The time-series duty ratios are associated with danger information indicating that there is a risk of occurrence of a failure in the infusion pump 1 after the prediction period. In addition, the training data includes a plurality of time-series duty ratios obtained from the infusion pump 1 in which no failure has occurred. The time-series duty ratios are associated with danger information indicating that there is no risk of occurrence of a failure in the infusion pump 1 after the prediction period.

In the machine learning, the learning device inputs the flow rate and the time-series duty ratio included in the training data to a model to be a base of the learned model 432, and the model performs calculation according to the input of the flow rate and the time-series duty ratio and outputs danger information. The learning device adjusts parameters of calculation of the model so as to reduce an error between the danger information output by the model and the danger information associated with the input time-series duty ratio. For example, in a case in which a time-series duty ratio obtained from the infusion pump 1 in which a failure has occurred is input, the parameters are adjusted such that the model outputs danger information indicating that the probability of occurrence of a failure is almost 100%. For example, in a case in which a time-series duty ratio obtained from the infusion pump 1 in which no failure has occurred is input, the parameters are adjusted such that the model outputs danger information indicating that the probability of occurrence of a failure is almost 0%.

The learning device performs machine learning by repeating processing using a plurality of sets of the flow rate, the time-series duty ratio, and the danger information included in the training data and adjusting parameters of the model. The learned model 432 is generated by adjusting the parameters of the calculation in this manner. For example, the adjusted final parameters are stored in the storage unit 43, and the calculation unit 41 executes information processing using the parameters, thereby the learned model 432 is implemented.

FIG. 15 is a flowchart indicating an example of procedure of processing to be executed by the information processing system according to the third embodiment. The information processing system executes the processing from S201 to S214 in a similar manner to the second embodiment. In S206, in a case in which the second average value is equal to or less than the second upper limit value and equal to or greater than the second lower limit value (S206: No), the information processing apparatus 4 inputs the flow rate of the medicinal solution to be delivered by the infusion pump 1 and the time-series duty ratio of the voltage to be applied to the motor 141 of the infusion pump 1 to the learned model 432 (S31).

The information processing apparatus 4 receives the flow rate transmitted from the infusion pump 1 in advance by the communication unit 47 and stores the flow rate in the storage unit 43. In S31, the calculation unit 41 inputs the flow rate stored in the storage unit 43 and the latest time-series duty ratio included in the history of the duty ratio stored in the storage unit 43 to the learned model 432 and causes the learned model 432 to execute processing. The number of duty ratios to be included in the time-series duty ratio is determined in advance. The learned model 432 outputs danger information in response to the input of the flow rate and the time-series duty ratio.

The learned model 432 to be used in S31 may have a form in which the temperature is input in addition to the flow rate and the time-series duty ratio. In this form, the learned model 432 is generated by machine learning using training data including the time-series duty ratio and the flow rate and the temperature when the time-series duty ratio is obtained. The learned model 432 is learned in advance so as to output danger information regarding a risk of occurrence of a failure in the infusion pump 1 in a case in which the flow rate, the temperature, and the time-series duty ratio are input. For example, the infusion pump 1 includes a temperature sensor and transmits the measured temperature to the information processing apparatus 4. In S31, the information processing apparatus 4 inputs the received temperature, flow rate, and time-series duty ratio to the learned model 432. The learned model 432 outputs danger information in response to input of the flow rate, the temperature, and the time-series duty ratio.

The information processing apparatus 4 acquires the danger information output by the learned model 432 (S32). Next, the information processing apparatus 4 determines whether or not there is a risk of occurrence of a failure in the infusion pump 1 based on the acquired danger information (S33). For example, the calculation unit 41 acquires the danger information in S32, and in S33, in a case in which the acquired danger information indicates that there is a risk of occurrence of a failure, it is determined that there is a risk of occurrence of a failure, and in a case in which the acquired danger information indicates that there is no risk of occurrence of a failure, it is determined that there is no risk of occurrence of a failure. For example, in S33, the calculation unit 41 determines that there is a risk of occurrence of a failure in a case in which a probability indicated by the danger information exceeds a predetermined threshold value, and determines that there is no risk of occurrence of a failure in a case in which the probability indicated by the danger information is equal to or less than the threshold value.

In a case in which it is determined that there is no risk of occurrence of a failure (S33: No), the information processing apparatus 4 ends the processing. In a case in which it is determined that there is a risk of occurrence of a failure (S33: Yes), the information processing apparatus 4 outputs prediction that a failure will occur in the infusion pump 1 after the prediction period (S34). In S34, the calculation unit 41 displays an image including a sentence indicating that it is predicted that a failure will occur in the infusion pump 1 after the prediction period on the display unit 45. In addition, the calculation unit 41 causes the communication unit 47 to transmit data indicating that it is predicted that the failure will occur to the display device 5. The display device 5 displays an image indicating that it is predicted that the failure will occur based on the data from the information processing apparatus 4. The calculation unit 41 may generate sound indicating prediction that the failure will occur in the infusion pump 1 from the sound generation unit 46. The processing in S34 corresponds to an output unit.

The information processing apparatus 4 may output the probability indicated by the danger information in S34. In this form, the calculation unit 41 displays an image including the probability of occurrence of a failure in the infusion pump 1 after the prediction period indicated by the danger information on the display unit 45 and the display device 5. In the mode in which the probability indicated by the danger information is output, the information processing apparatus 4 may omit the processing in S33. After the processing in S34 ends, the information processing apparatus 4 ends the processing. While the infusion pump 1 is operating, the processing from S201 to S214 and from S31 to S34 is repeatedly executed.

In the above description, a mode in which only the determination using the learned model 432 is performed to predict a failure of the infusion pump 1 has been described. However, a mode may be employed where the information processing apparatus 4 executes both determination using the learned model 432 and determination based on comparison between the first average value and the first range similar to the second embodiment to predict a failure of the infusion pump 1. For example, the information processing apparatus 4 may perform processing of determining that there is a risk of occurrence of a failure in a case in which it is determined that there is a risk of occurrence of a failure in both determination using the learned model 432 and determination based on comparison between the first average value and the first range. In a case in which it is determined that there is a risk of occurrence of a failure in at least one of determination using the learned model 432 or determination based on comparison between the first average value and the first range, the information processing apparatus 4 may perform processing of determining that there is a risk of occurrence of a failure. The information processing apparatus 4 may output both a result of determination using the learned model 432 and a result of determination based on comparison between the first average value and the first range.

As described above in detail, the infusion pump 1 performs liquid delivery while maintaining a constant flow rate, and the information processing apparatus 4 determines whether or not there is a risk of a failure using the learned model 432 based on the duty ratio of the voltage to be applied to the motor 141 of the infusion pump 1. By using the learned model 432, it is possible to easily determine whether or not there is a risk of occurrence of a failure. Also in the third embodiment, it is possible to predict occurrence of a failure in the infusion pump 1 before the failure occurs, and the user can easily recognize a risk of occurrence of a failure. Maintenance of the infusion pump 1 becomes more efficient than the related art. In addition, it is possible to avoid occurrence of a failure in the infusion pump 1, and safety of treatment is improved.

In the third embodiment, the mode in which the information processing apparatus 4 predicts a failure of the infusion pump 1 using the learned model 432 has been described. However, the infusion pump 1 may predict a failure by using the learned model by itself. The infusion pump 1 may execute both determination using the learned model and determination based on comparison between the first average value and the first range. In the first to the third embodiments, a mode in which the infusion pump 1 is a PCA pump and delivers a medicinal solution has been described. However, as the infusion pump 1, a form used for applications other than the PCA pump may be used, or a form in which liquid other than a medicinal solution is delivered may be used.

The present invention is not limited to the content of the above-described embodiments, and various modifications can be made within the scope indicated in the claims. In other words, embodiments obtained by combining technical means appropriately changed within the scope indicated in the claims are also included in the technical scope of the present invention.

### REFERENCE SIGNS LIST

1 Infusion pump
11 Pump body
13 Control unit
14 Liquid delivery unit
141 Motor
21 Injection needle
22 Infusion tube
3 Patient
4 Information processing apparatus
41 Calculation unit
43 Storage unit
431 Computer program
432 Learned model
5 Display device

1. An information processing method comprising:
recording a duty ratio of a voltage to be applied to a motor included in an infusion pump that delivers liquid while maintaining a constant flow rate;
determining whether or not there is a risk of occurrence of a failure in the infusion pump based on a history of the duty ratio; and
in a case in which there is a risk of occurrence of a failure, outputting a prediction of occurrence of the failure.

2. The information processing method according to claim 1, comprising:
calculating a first average value by averaging duty ratios during a predetermined first period; and
determining whether or not there is a risk of occurrence of a failure in the infusion pump by determining whether or not the first average value is outside a specific first range.

3. The information processing method according to claim 2, comprising:
calculating a second average value by averaging duty ratios during a predetermined second period;
determining whether or not a failure has occurred in the infusion pump by determining whether or not the second average value is outside a predetermined second range;
in a case in which a failure has occurred in the infusion pump, outputting that the failure has occurred; and
in a case in which a failure has not occurred in the infusion pump, determining whether or not there is a risk of occurrence of a failure in the infusion pump.

4. The information processing method according to claim 3, comprising:
in a case in which a failure has occurred in the infusion pump, adjusting the first range based on the history of the duty ratio.

5. The information processing method according to claim 3, wherein the first period is longer than the second period.

6. The information processing method according to claim 2, wherein the first range is narrower as the flow rate is higher.

7. The information processing method according to claim 1, comprising:
determining whether or not there is a risk of occurrence of a failure in the infusion pump by:
inputting a recorded time-series duty ratio and a flow rate of liquid to be delivered by the infusion pump to a learned model that has learned to output information on a risk of occurrence of a failure in the infusion pump upon receiving an input comprising the time-series duty ratio and the flow rate of the liquid to be delivered by the infusion pump, and acquiring the information output by the learned model.

8. The information processing method according to claim 1, comprising:
calculating a cycle of temporal change of the duty ratio; and
specifying a portion where a failure has occurred in the infusion pump according to the cycle.

9. An infusion pump for delivering liquid while maintaining a constant flow rate, the infusion pump comprising:
a motor that operates to deliver the liquid;
a recording unit that records a duty ratio of a voltage to be applied to the motor to operate the motor;
a determination unit configured to determine whether or not there is a risk of occurrence of a failure in the infusion pump based on a history of the duty ratio; and
an output unit configured to, in a case in which there is a risk of occurrence of a failure, output a prediction of occurrence of the failure.

10. An information processing apparatus comprising:
an acquisition unit configured to acquire, from an infusion pump, a duty ratio of a voltage to be applied to a motor included in the infusion pump that delivers liquid while maintaining a constant flow rate;
a recording unit configured to record the acquired duty ratio;
a determination unit configured to determine whether or not there is a risk of occurrence of a failure in the infusion pump based on a history of the duty ratio; and
an output unit configured to, in a case in which there is a risk of occurrence of a failure, output a prediction of occurrence of the failure.

11. A computer program causing a computer to execute processing of:
Acquiring, from an infusion pump, a duty ratio of a voltage to be applied to a motor included in the infusion pump that delivers liquid while maintaining a constant flow rate; and
recording the acquired duty ratio;
determining whether or not there is a risk of occurrence of a failure in the infusion pump based on a history of the duty ratio; and
in a case in which there is a risk of occurrence of a failure, outputting prediction of occurrence of the failure.

12. An information processing system comprising:
an infusion pump; and
an information processing apparatus; wherein:
the infusion pump comprises a motor configured to operate to deliver liquid and to transmit, to the information processing apparatus, a duty ratio of a voltage to be applied to the motor to operate the motor; and
the information processing apparatus is configured to:
receive the duty ratio,
record the received duty ratio,
determine whether or not there is a risk of occurrence of a failure in the infusion pump based on a history of the duty ratio, and
in a case in which there is a risk of occurrence of a failure, output prediction of occurrence of the failure.
